(19) United States
(12) Patent Application Publication
(10) Pub. No.: US 2026/0224238 A1
(43) Pub. Date: Aug. 6, 2026

(54) ULTRASOUND TREATMENT TOOL

(71) Applicant: OLYMPUS MEDICAL SYSTEMS CORP., Tokyo (JP)

(72) Inventors: Kaito SATO, Tokyo (JP); Tsubasa NIIYAMA, Tokyo (JP)

(21) Appl. No.: 19/635,132

(22) Filed: Mar. 31, 2026

Related U.S. Application Data
(63) Continuation of application No. PCT/JP2024/034527, filed on Sep. 26, 2024.
(60) Provisional application No. 63/587,269, filed on Oct. 2, 2023.

(51) Int. Cl. A61B 17/32

(52) U.S. Cl. CPC A61B 17/320092; A61B 2017/320095

(57) ABSTRACT

An ultrasound treatment tool includes: a vibration transmitter configured to transmit ultrasound vibration from a proximal end toward a distal end; a tube into which the vibration transmitter is inserted; and a rigid part that is provided on an outer peripheral surface of the vibration transmitter at a node position of the ultrasound vibration, the rigid part being in contact with an inner peripheral surface of the tube, the rigid part being made of a resin material that is harder than the tube.

### CROSS-REFERENCE TO RELATED APPLICATION

This application is a continuation of International Application No. PCT/JP2024/034527, filed on September 26, 2024 which claims the benefit of priority of U.S. Provisional Application No. 63/587,269 filed on October 2, 2023, the entire contents of which are incorporated herein by reference.

### BACKGROUND

### 1. Technical Field

The present disclosure relates to an ultrasound treatment tool.

### 2. Related Art

In the related art, there is a known ultrasound treatment tool that performs treatment on a region targeted for treatment of biological tissue (hereinafter, referred to as a treatment target) by applying ultrasound energy as treatment energy to the treatment target (for example, see Japanese Laid-open Patent Publication No. 2022-2807).

In the ultrasound treatment tool described in Patent Literature 1, a vibration transmitter (ultrasound blade), a jaw (jaw portion), a rubber lining, and a pipe (inner side sheath) described below are provided.

The vibration transmitter transmits ultrasound vibration from a proximal end toward a distal end.

The jaw is opened and closed with respect to the vibration transmitter.

The rubber lining has a circular ring shape, and is provided on an outer peripheral surface of the vibration transmitter. More specifically, the rubber lining is provided at a node position of the ultrasound vibration.

In the pipe, the vibration transmitter is inserted, and an outer peripheral surface of the rubber lining abuts against an inner peripheral surface of the pipe.

### SUMMARY

In some embodiments, an ultrasound treatment tool includes: a vibration transmitter configured to transmit ultrasound vibration from a proximal end toward a distal end; a tube into which the vibration transmitter is inserted; and a rigid part that is provided on an outer peripheral surface of the vibration transmitter at a node position of the ultrasound vibration, the rigid part being in contact with an inner peripheral surface of the tube, the rigid part being made of a resin material that is harder than the tube, the rigid part being a pair of C rings each having a C-shape into which the vibration transmitter is inserted, each C ring including a C ring main body having a C-shape in which a notch is formed, and a protrusion protruding from the C ring main body along a longitudinal direction of the vibration transmitter, the pair of C rings being attached to the vibration transmitter in a state in which the protrusion of one of the pair of C rings is arranged at the notch of another one of the pair of C rings.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating a treatment system according to an embodiment;

FIG. 2 is a diagram explaining a configuration a distal end portion of an ultrasound treatment tool;

FIG. 3 is a diagram illustrating a configuration of a support;

FIG. 4 is a diagram explaining a configuration of a first support;

FIGS. 5A, 5B and 5C are diagrams explaining a configuration of the first support;

FIG. 6 is a diagram explaining a watertight structure constituted by the first support;

FIG. 7 is a diagram explaining the watertight structure constituted by the first support;

FIG. 8 is a diagram explaining the watertight structure constituted by the first support;

FIGS. 9A, 9B and 9C are diagrams explaining a first modification of the embodiment;

FIG. 10 is a diagram explaining the first modification of the embodiment;

FIG. 11 is a diagram explaining the first modification of the embodiment;

FIG. 12 is a diagram explaining a second modification of the embodiment;

FIGS. 13A, 13B and 13C are diagrams explaining a third modification of the embodiment;

FIG. 14 is a diagram explaining the third modification of the embodiment;

FIG. 15 is a diagram explaining the third modification of the embodiment;

FIGS. 16A and 16B are diagrams explaining a fourth modification of the embodiment;

FIG. 17 is a diagram explaining the fourth modification of the embodiment; and

FIG. 18 is a diagram explaining the fourth modification of the embodiment.

### DETAILED DESCRIPTION

Hereinafter, modes (hereinafter, embodiments) for carrying out the present invention will be described below with reference to the drawings. Furthermore, the present invention is not limited to the embodiments described below. In addition, in description of the drawings, components that are identical to those in drawings are assigned the same reference numerals.

### Schematic configuration of treatment system

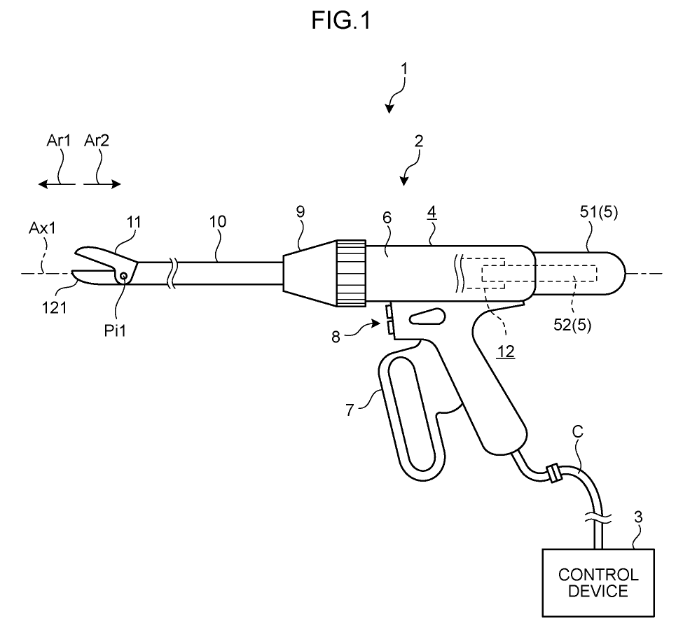

FIG. 1 is a diagram illustrating a treatment system 1 according to an embodiment.

The treatment system 1 performs treatment on a region targeted for treatment of biological tissue (hereinafter, referred to as a treatment target) by applying treatment energy to the treatment target. The treatment energy described in the present embodiment is ultrasound energy and high frequency energy. Furthermore, the treatment that is able to be performed by the treatment system 1 according to the present embodiment is treatment including coagulation (sealing) of the treatment target, incision of the treatment target, or the like. Furthermore, both of the coagulation and the incision may be performed at the same time. The treatment system 1 includes, as illustrated in FIG. 1, an ultrasound treatment tool 2 and a control device 3.

### Configuration of ultrasound treatment tool

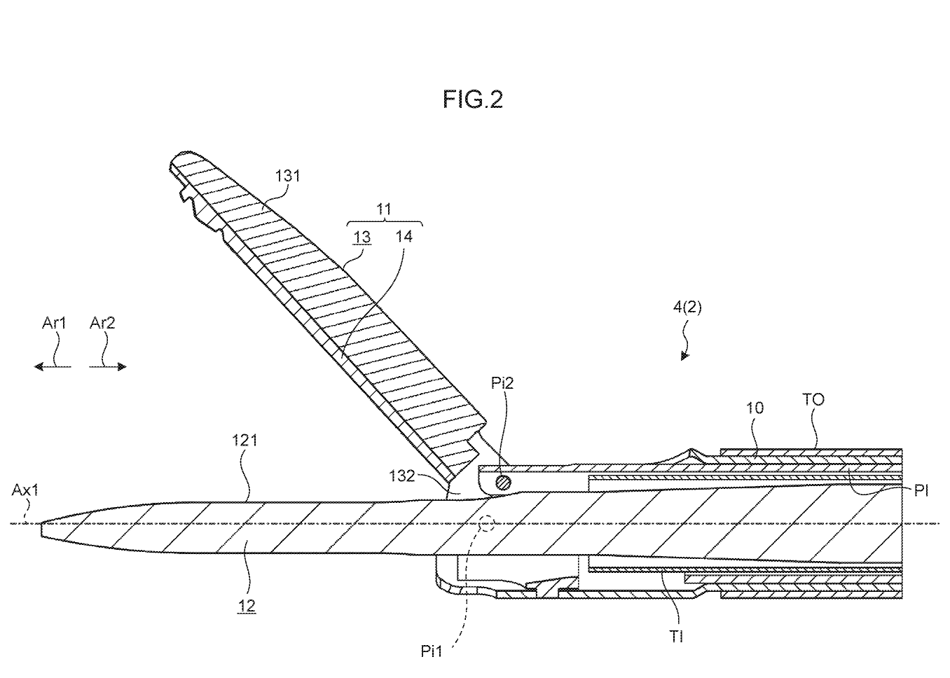

Moreover, in the following description, one of the sides along a central axis Ax1 (FIG. 1) of an outer pipe 10 is referred to as a distal end side Ar1, whereas the other of the sides is referred to as a proximal end side Ar2. In addition, a "width direction" described below is a direction perpendicular to the opening/closing direction of a jaw 11 with respect to the central axis Ax1 and a treatment portion 121, and indicates the direction perpendicular to the planes of the drawings in FIG. 1 and FIG. 2.

FIG. 2 is a diagram explaining a configuration of a distal end portion of the ultrasound treatment tool 2. Specifically, FIG. 2 is a cross-sectional view of the distal end portion of the ultrasound treatment tool 2 obtained by cutting the distal end portion of the ultrasound treatment tool 2 by a plane that includes the central axis Ax1 of the outer pipe 10 in a state in which the jaw 11 and a vibration transmitter 12 are included in the plane.

The ultrasound treatment tool 2 is a treatment tool that performs treatment on the treatment target by applying ultrasound energy and high frequency energy to the treatment target. The ultrasound treatment tool 2 includes, as illustrated in FIG. 1, a handpiece 4 and an ultrasound transducer unit 5.

The handpiece 4 includes, as illustrated in FIG. 1 and FIG. 2, a fixed handle 6 (FIG. 1), an operation handle 7 (FIG. 1), a switch 8 (FIG. 1), a rotatable knob 9 (FIG. 1), the outer pipe 10, the jaw 11, and the vibration transmitter 12.

The fixed handle 6 is a portion that supports the entire of the ultrasound treatment tool 2, and that is gripped by an operator (user), such as an operating person.

The operation handle 7 is attached to the fixed handle 6 such that the operation handle 7 is able to move with respect to the fixed handle 6, and receives an opening and closing operation performed by the operator, such as the operating person.

The switch 8 is provided such that the switch 8 is exposed to the outside from the fixed handle 6, and receives a treatment operation performed by the operator, such as the operating person.

The rotatable knob 9 is formed to have a substantially cylindrical shape that is coaxial with the central axis Ax1, and is provided at the fixed handle 6 on the distal end side Ar1. Then, the rotatable knob 9 receives a rotation operation performed by the operator, such as the operating person. As a result of the rotation operation, the rotatable knob 9 rotates about the central axis Ax1 relative to the fixed handle 6. Furthermore, as a result of the rotation of the rotatable knob 9, the outer pipe 10, the jaw 11, and the vibration transmitter 12 also rotate about the central axis Ax1.

The outer pipe 10 has a tube shape. In the present embodiment, the outer pipe 10 is a pipe that is formed in a cylindrical shape, and that is made of an electrically conductive material, such as metal.

In the outer pipe 10, a pivot axis Pi1 (FIG. 1 and FIG. 2) that has a columnar shape, that extends in the direction perpendicular to the planes of the drawings in FIG. 1 and FIG. 2, and that is engaged with the jaw 11 and pivotally and rotatably supports the jaw 11 is fixed at the end portion of the outer pipe 10 located on the distal end side Ar1.

Then, the outer peripheral surface of the outer pipe 10 is covered by an outer tube TO (FIG. 2) having an electrical insulating property. Furthermore, an inner pipe PI (FIG. 2) that is formed in a tube shape and that moves forward and backward along the longitudinal direction of the outer pipe 10 in accordance with the opening and closing operation performed on the operation handle 7 by the operator, such as the operating person, is inserted in the interior of the outer pipe 10. The inner pipe PI corresponds to a pipe. Then, a drive axis Pi2 (FIG. 2) that has a columnar shape extending in the direction perpendicular to the planes of the drawings in FIG. 1 and FIG. 2, and that is engaged with the jaw 11 is fixed to the end portion of the inner pipe PI located on the distal end side Ar1. In the present embodiment, the drive axis Pi2 is arranged on the upper side of the pivot axis Pi1 in FIG. 2 (on the side in which an arm main body 131 is arranged with respect to the treatment portion 121).

In the present embodiment, a part of the jaw 11 is made of an electrically conductive material. The jaw 11 includes, as illustrated in FIG. 2, an arm 13 and a pad 14.

In the present embodiment, the arm 13 is made of an electrically conductive material. The arm 13 is a member that is integrally formed of, as illustrated in FIG. 2, the arm main body 131 and a pair of bearings 132.

The arm main body 131 is configured by use of a plate body having a long shape, and one of the plate surfaces is arranged in a posture in which the one of the plate surfaces faces the vibration transmitter 12.

Each of the pair of bearings 132 is provided at the end portion of the arm main body 131 located on the proximal end side Ar2, and is configured by use of a plate body that faces the width direction of the arm main body 131. Moreover, in FIG. 2, only the portion corresponding to one of the bearings 132 between the pair of bearings 132 is illustrated. The pair of bearings 132 have the same configuration, so that, in the following description, only a configuration of one of the bearings 132 will be described.

The bearing 132 is connected to the outer pipe 10 by the pivot axis Pi1. Furthermore, the bearing 132 is connected to the inner pipe PI by the drive axis Pi2.

Then, the arm 13 pivots around the pivot axis Pi1 with respect to the outer pipe 10 in conjunction with forward and backward movements of the inner pipe PI in accordance with the opening and closing operation performed on the operation handle 7 by the operator, such as the operating person. As a result of this, the jaw 11 is opened and closed with respect to the treatment portion 121 that is the end portion of the vibration transmitter 12 located on the distal end side, and enables the treatment target to be gripped between the treatment portion 121 and the jaw 11. In the present embodiment, the jaw 11 pivots around the pivot axis Pi1 in a direction closer to the treatment portion 121 in conjunction with the movement of the jaw 11 to the inner pipe PI that is located on the distal end side Ar1. In other words, the jaw 11 is closed with respect to the treatment portion 121. Furthermore, the jaw 11 pivots around the pivot axis Pi1 in a direction away from the treatment portion 121 in conjunction with the movement of the jaw 11 to the inner pipe PI that is located on the proximal end side Ar2. In other words, the jaw 11 is opened with respect to the treatment portion 121. As described above, in the present embodiment, the ultrasound treatment tool 2 is configured to have, what is called, a push close type.

The pad 14 is made of a resin material having an electrical insulating property and biological compatibility, such as, polytetrafluoroethylene (PTFE), and has a substantially rectangular parallelepiped shape along the longitudinal direction of the arm 13. Then, the pad 14 is fixed to, as illustrated in FIG. 2, the surface of the arm main body 131 located on the treatment portion 121 side, and abuts against the treatment portion 121 when the jaw 11 is closed with respect to the treatment portion 121. The pad 14 has the electrical insulating property, and accordingly has a function of preventing the arm 13 and the vibration transmitter 12 from being short circuited. Furthermore, the pad 14 has a function to prevent the treatment portion 121 that is generating ultrasound vibration from being damaged by hitting against the arm 13 when incision of the treatment target performed by using the ultrasound vibration has been completed.

Moreover, an example of the fixing method of fixing the pad 14 with respect to the arm main body 131 includes, for example, a fixing method of protruding a claw portion from the surface of the arm main body 131 located on the treatment portion 121 side and mechanically fixing by locking the pad 14 to the claw portion, a method of insert molding, or the like.

The vibration transmitter 12 is made of an electrically conductive material, and has a long shape extending along the central axis Ax1. Furthermore, the vibration transmitter 12 is, as illustrated in FIG. 2, inserted into the interior of inner pipe PI in a state in which the treatment portion 121 protrudes outside. At this time, the end portion of the vibration transmitter 12 located on the proximal end side Ar2 is, as illustrated in FIG. 1, mechanically connected to an ultrasound transducer 52 that constitutes the ultrasound transducer unit 5. Then, the vibration transmitter 12 transmits the ultrasound vibration that has been generated by the ultrasound transducer unit 5 from the end portion of the vibration transmitter 12 located on the proximal end side Ar2 to the treatment portion 121. In the present embodiment, the ultrasound vibration is longitudinal vibration that vibrates in a direction along the central axis Ax1. Furthermore, the outer peripheral surface of the vibration transmitter 12 other than the treatment portion 121 is covered by an inner tube TI (FIG. 2) that has an electrical insulating property. As the inner tube TI, a heat shrink tube that is made of a fluororesin, such as perfluoroethylene propylene copolymer (FEP) may be used. The inner tube TI corresponds to a tube.

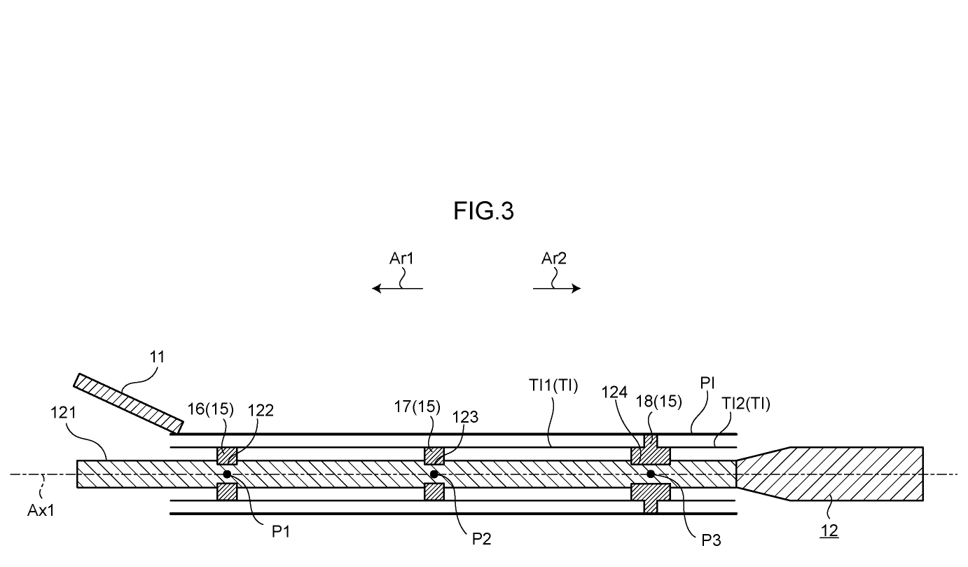

On an outer peripheral surface of the vibration transmitter 12 that has been described above, a support 15 (see FIG. 3) is provided.

Moreover, a detailed configuration of the support 15 will be explained later in a "configuration of support" that will be described later.

The ultrasound transducer unit 5 includes, as illustrated in FIG. 1, a transducer (TD) case 51 and the ultrasound transducer 52.

The TD case 51 supports the ultrasound transducer 52, and is connected to the fixed handle 6 so as to be capable of being attached to and removed from the fixed handle 6.

The ultrasound transducer 52 generates ultrasound vibration under the control of the control device 3. In the present embodiment, the ultrasound transducer 52 is constituted by a bolt-clamped Langevin-type transducer (BLT).

### Configuration of control device

The control device 3 performs overall control of the operation of the ultrasound treatment tool 2 by way of an electric cable C (FIG. 1).

Specifically, the control device 3 detects the treatment operation performed on the switch 8 by the operator, such as the operating person, by way of the electric cable C. Then, when the control device 3 has detected the treatment operation, the control device 3 applies, by way of the electric cable C, treatment energy to the treatment target that is gripped between the jaw 11 and the treatment portion 121. In other words, the control device 3 performs treatment on the treatment target.

For example, when the control device 3 applies ultrasound energy to the treatment target, the control device 3 supplies driving electrical power to the ultrasound transducer 52 by way of the electric cable C. As a result of this, the ultrasound transducer 52 generates longitudinal vibration (ultrasound vibration) that vibrates in the direction along the central axis Ax1. Furthermore, the treatment portion 121 vibrates at a desired amplitude by the longitudinal vibration. Then, the ultrasound vibration is applied from the treatment portion 121 to the treatment target that is gripped between the jaw 11 and the treatment portion 121. In other words, the ultrasound energy is applied from the treatment portion 121 to the treatment target.

Furthermore, for example, when high frequency energy is applied to the treatment target, the control device 3 supplies high frequency electrical power between the arm 13 and the vibration transmitter 12 by way of the electric cable C, the outer pipe 10, and the like. Then, when the high frequency electrical power is supplied between the arm 13 and the vibration transmitter 12, a high frequency current flows into the treatment target that is gripped between the jaw 11 and the treatment portion 121. In other words, the high frequency energy is applied to the treatment target. In other words, the arm 13 and the treatment portion 121 function as electrodes.

### Configuration of support

In the following, a configuration of the above described support 15 will be explained.

FIG. 3 is a diagram illustrating the configuration of the support 15. Specifically, FIG. 3 is a cross-sectional view obtained by cutting the jaw 11, the vibration transmitter 12, the inner pipe PI, the inner tube TI, and the support 15 by the same plane as that described above in FIG. 2.

The support 15 includes, as illustrated in FIG. 3, first to third supports 16 to 18.

In the present embodiment, a third support 18 is made of an elastic rubber, and has a circular ring shape into which the vibration transmitter 12 is inserted. More specifically, the third support 18 is disposed at a node position P3 (FIG. 3) that is located at a position closest to the proximal end side Ar2 from among the node positions of the ultrasound vibration (longitudinal vibration). Then, the third support 18 is contact with the inner peripheral surface of the inner pipe PI. Furthermore, in the present embodiment, the inner tube TI is, as illustrated in FIG. 3, divided into two portions by the third support 18. In the following description, between the two portions, the inner tube TI that is located at a position closer to the distal end side Ar1 than the third support 18 is referred to as a first inner tube TI1, and the inner tube TI that is located at a position closer to the proximal end side Ar2 than the third support 18 is referred to as a second inner tube TI2 (FIG. 3).

Each of the first and the second supports 16 and 17 is made of a resin material that is harder than the inner tube TI, and has a circular ring shape into which the vibration transmitter 12 is inserted. Examples of the resin material include polytetrafluoroethylene (PTFE), polyether ether ketone (PEEK), polyphenylsulfone (PPSU), and polyoxymethylene (POM). More specifically, the first support 16 is disposed at a node position P1 (FIG. 3) that is located at a position closest to the distal end side Ar1 from among the node positions of the ultrasound vibration (longitudinal vibration). The first support 16 corresponds to a rigid part. Furthermore, the second support 17 is disposed at a node position P2 (FIG. 3) that is located at a position second closest to the distal end side Ar1 from among the node positions of the ultrasound vibration (longitudinal vibration). The second support 17 corresponds to the rigid part.

Here, as illustrated in FIG. 3, grooves 122 to 124 each having a circular ring shape and in which the first to the third supports 16 to 18 are respectively attached are provided on the outer peripheral surface of the vibration transmitter 12 at the respective node positions P1 to P3.

Moreover, the first and the second supports 16 and 17 have the same configuration. Therefore, in the following description, only a configuration of the first support 16 will be described.

### Configuration of first support

In the following, a configuration of the above described first support 16 will be described.

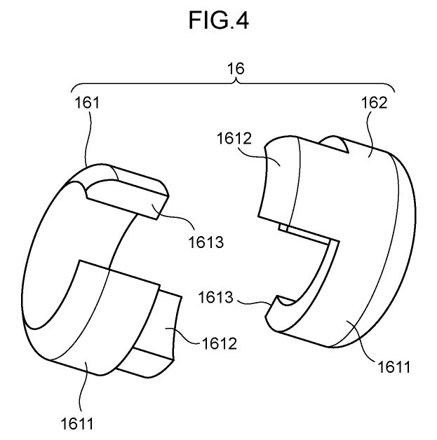

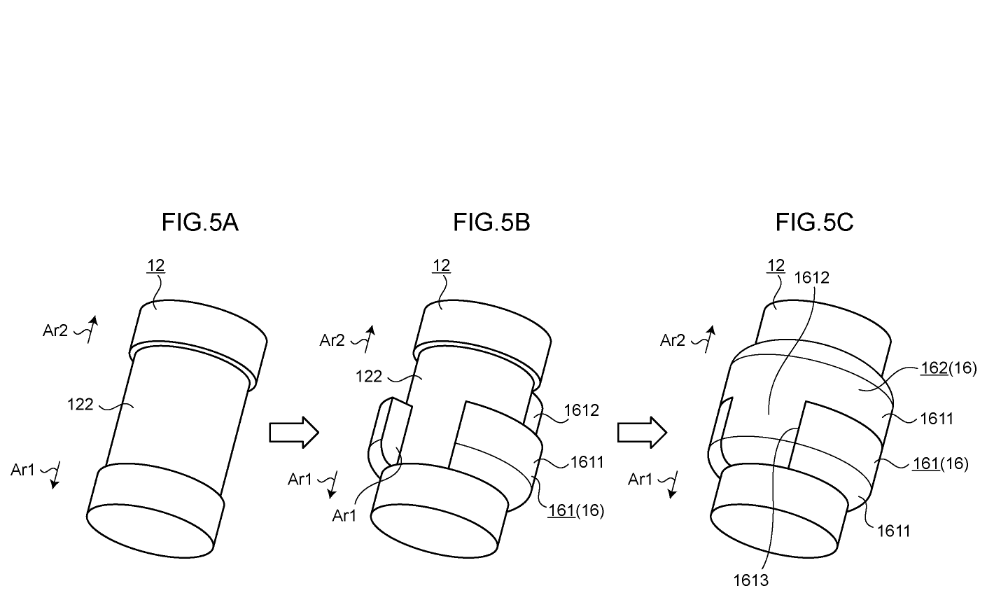

FIG. 4 and FIGS. 5A, 5B and 5C are diagrams each explaining the configuration of the first support 16. Specifically, FIG. 4 is an exploded perspective view illustrating the first support 16. FIGS. 5A, 5B and 5C are diagrams illustrating an assembly order of the first support 16 with respect to the vibration transmitter 12.

The first support 16 is configured, as illustrated in FIG. 4 and FIGS. 5A, 5B and 5C, a first and a second C rings 161 and 162.

The first and the second C rings 161 and 162 have the same shape. Therefore, in the following, only the configuration of the first C ring 161 will be described, and regarding the second C ring 162, components that are the same as those included in the first C ring 161 are assigned the same reference numerals and descriptions thereof in detail will be omitted.

The first C ring 161 includes, as illustrated in FIG. 4, a C ring main body 1611 and a protrusion 1612.

The C ring main body 1611 is a member that has a C-shape that has been formed as a result of a notch 1613 being formed at a part of circumferential direction of a circular ring that has substantially the same inner diameter dimension as the outer diameter dimension of the groove 122. Moreover, it is preferable that the inner diameter dimension of the C ring main body 1611 is constituted to have a dimension that is smaller than the outer diameter dimension of the groove 122 in order to constitute the structure in which a gap is not formed between the C ring main body 1611 and the vibration transmitter 12.

The protrusion 1612 is a portion that protrudes along the central axis of the C ring main body 1611 that is rotationally symmetrical at 180° about the central axis of the C ring main body 1611 with respect to a place in which the notch 1613 is provided on the C ring main body 1611.

Then, the first support 16 is assembled, as described below, in the groove 122.

Specifically, first, a worker arranges the vibration transmitter 12 on an inner side of the C ring main body 1611 formed in a C-shape while pushing out both end portions of the circumferential direction of the C ring main body 1611 included in the first C ring 161 (both edge portions of the notch 1613) in a direction away from each other (FIG. 5B). Furthermore, the worker arranges, in a similar manner, the vibration transmitter 12 on an inner side of the C ring main body 1611 formed in the C-shape while pushing out both end portions of the circumferential direction of the C ring main body 1611 included in the second C ring 162 (both edge portions of the notch 1613) in a direction away from each other in a state in which the protrusion 1612 included in the second C ring 162 fits into the notch 1613 included in the first C ring 161 (FIG. 5C). In other words, the first and the second C rings 161 and 162 are attached to the vibration transmitter 12 by using, what is called, a snap-fit method.

### Watertight structure constituted by first support

In the following, a watertight structure constituted by the first support 16 will be described.

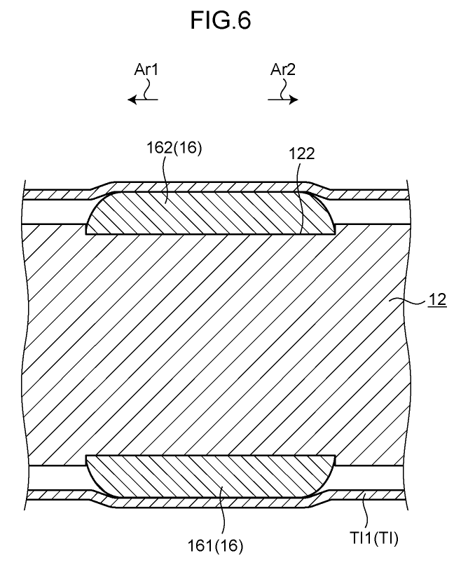

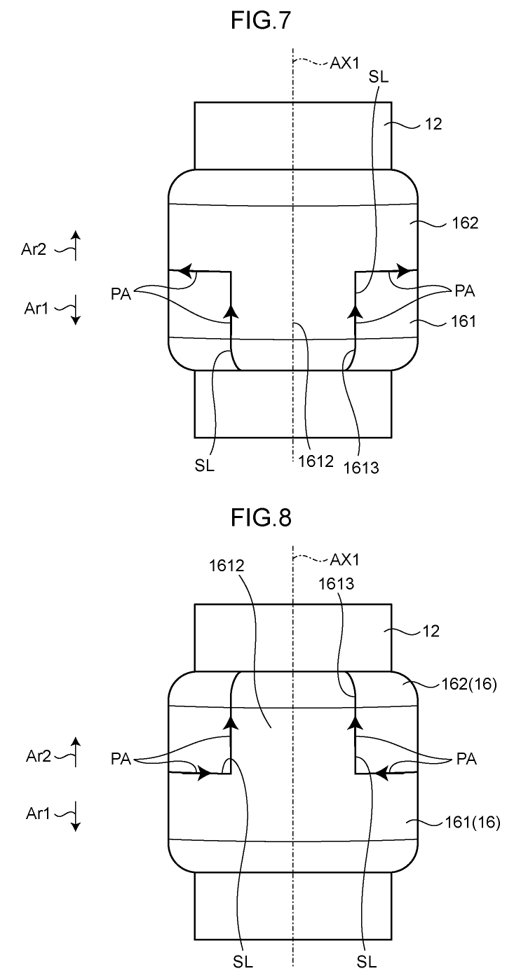

FIG. 6 and FIG. 8 are diagrams each illustrating a watertight structure constituted by the first support 16. Specifically, FIG. 6 is a cross-sectional view obtained by cutting the first inner tube TI1, the first support 16, and the vibration transmitter 12 by a plane that includes the central axis Ax1. FIG. 7 and FIG. 8 are diagrams each illustrating paths PA for a fluid flowing from distal end side Ar1 toward the proximal end side Ar2 by way of the first support 16. Moreover, in FIG. 7 and FIG. 8, each of the paths PA is represented by an arrow that indicates a travelling direction of the fluid.

As described above, the outer peripheral surface of the first support 16 that has been assembled into the vibration transmitter 12 is in contact with the inner peripheral surface of the first inner tube TI1, as illustrated in FIG. 6. As a result of this, the fluid that has entered from the distal end of the inner pipe PI into the gap between the first inner tube TI1 and the vibration transmitter 12 is inhibited from flowing toward the proximal end side Ar2 by the first support 16. Specifically, when the fluid flows by way of the first support 16, the fluid follows each of boundary portions SL located between the first and the second C rings 161 and 162 (FIG. 7 and FIG. 8). Each of the boundary portions SL extends from one end to the other end of the first support 16 in the direction along the central axis Ax1, and corresponds to a slit. In other words, the boundary portions SL function as the respective paths PA for the fluid (FIG. 7 and FIG. 8). In the present embodiment, a part of each of the boundary portions SL intersects (perpendicular) with respect to the direction along the central axis Ax1.

According to the present embodiment explained above, the following effects are provided.

In the ultrasound treatment tool 2 according to the present embodiment, the first support 16 is made of the resin material that is harder than the inner tube T. As a result of this, as compared with the configuration conventionally constituted by using a rubber lining, it is possible to reduce an amount of deformation of the first support 16 in a case where the jaw 11 is closed, and it is thus possible to suppress a deflection of the vibration transmitter 12. In other words, it is possible to reliably grip the treatment target between the jaw 11 and the vibration transmitter 12.

Furthermore, in the ultrasound treatment tool 2, as described above, the watertight structure is implemented by the inner tube TI and the first support 16. As a result of this, it is possible to suppress the fluid that has entered from the distal end of the inner pipe PI from flowing into the gap between the first inner tube TI1 and the vibration transmitter 12 from flowing toward the proximal end side Ar2.

Therefore, with the ultrasound treatment tool 2 according to the present embodiment, it is possible to ensure watertightness while suppressing a deflection of the vibration transmitter 12.

Furthermore, in the ultrasound treatment tool 2 according to the present embodiment, the first support 16 is configured by the first and the second C rings 161 and 162 described above.

As a result of this, it is possible to attach the first and the second C rings 161 and 162 to the vibration transmitter 12 by using, what is called, the snap-fit method, and it is possible to improve ability to assemble the ultrasound treatment tool 2. Furthermore, it is possible to reduce assembly time, and it is possible to eliminate an operation of removing a burr generated in a conventional case in which a rubber lining is used, so that it is possible to improve a cost reduction of the ultrasound treatment tool 2. In addition, the first support 16 is made of a resin material, so that it is possible to narrow down a gap of the boundary portion SL located between the first and the second C rings 161 and 162, it is possible to constitute each of the paths PA to have a long and complicated shape, and it is possible to sufficiently ensure watertightness. In particular, a part of the boundary portion SL intersects (perpendicular to) a direction along the central axis Ax1, so that it is possible to effectively suppress the fluid from entering. Furthermore, by manufacturing the first support 16 that is made of the resin material by mold injection, a 3D printer, or the like, it is possible to manufacture the first support 16 at low cost.

Furthermore, in the ultrasound treatment tool 2 according to the present embodiment, the second support 17 has the same configuration as that of the first support 16.

As a result of this, it is possible to more preferably implement the effects of ensuring watertightness while suppressing a deflection of the vibration transmitter 12.

### Other embodiments

In the above, descriptions of the preferred embodiments have been described, but the present invention is not limited to only the embodiments described above.

In the above described embodiment, the ultrasound treatment tool 2 uses ultrasound energy and high frequency energy as the treatment energy that is applied to the treatment target, but the treatment energy is not limited to this example, and the ultrasound treatment tool 2 may use only the ultrasound energy.

In the above described embodiment, the ultrasound treatment tool 2 is configured to have, what is called, a push close type, but the configuration is not limited to this example, and the ultrasound treatment tool 2 may be configured to have, what is called, a pull close type. Specifically, the jaw 11 pivots around the pivot axis Pi1 in a direction closer to the treatment portion 121 in conjunction with the movement of the inner pipe PI to the proximal end side Ar2. In other words, the jaw 11 is closed with respect to the treatment portion 121. Furthermore, the jaw 11 pivots around the pivot axis Pi1 in a direction away from the treatment portion 121 in conjunction with the movement of the inner pipe PI to the distal end side Ar1. In other words, the jaw 11 is opened with respect to the treatment portion 121.

In the above described embodiment, the jaw 11 is opened and closed with respect to the treatment portion 121 in conjunction with the forward and backward movements of the inner pipe PI along the central axis Ax1, but the configuration is not limited to this example. A configuration in which the jaw 11 is opened and closed with respect to the treatment portion 121 in conjunction with the forward and backward movements of the outer pipe 10 along the central axis Ax1 may be adopted.

In the above described embodiment, the third support 18 may also be made of a resin material, similarly to the first and the second supports 16 and 17.

Furthermore, in the above described embodiment, it may be possible to adopt a first to a fourth modifications that will be described below.

### First Modification

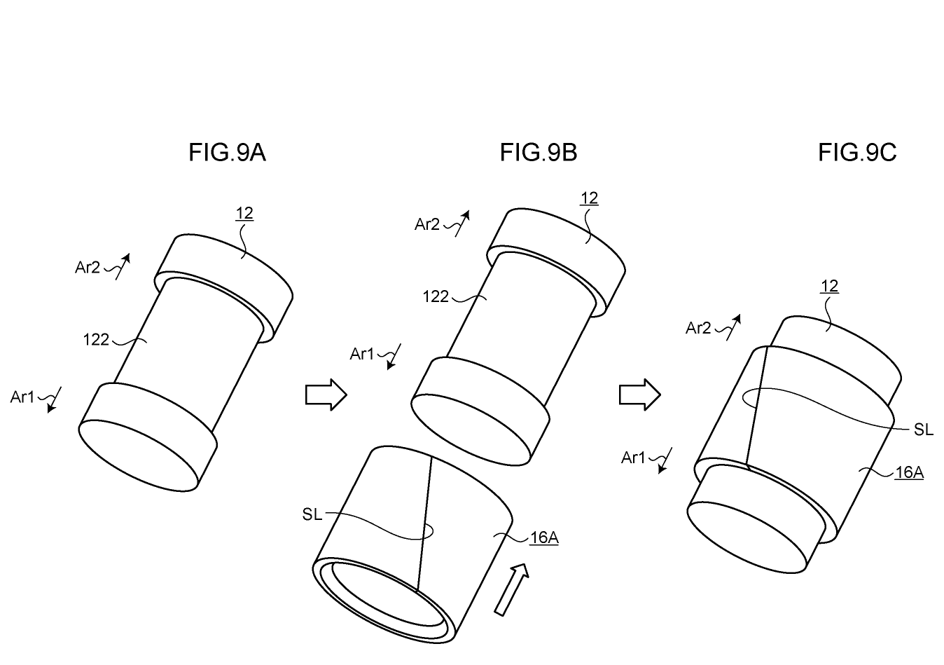

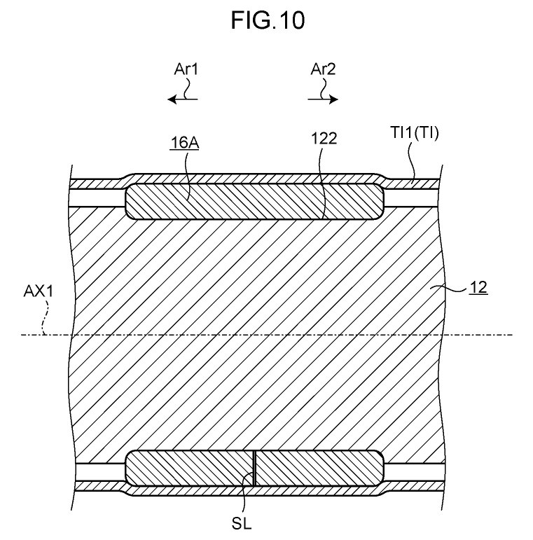

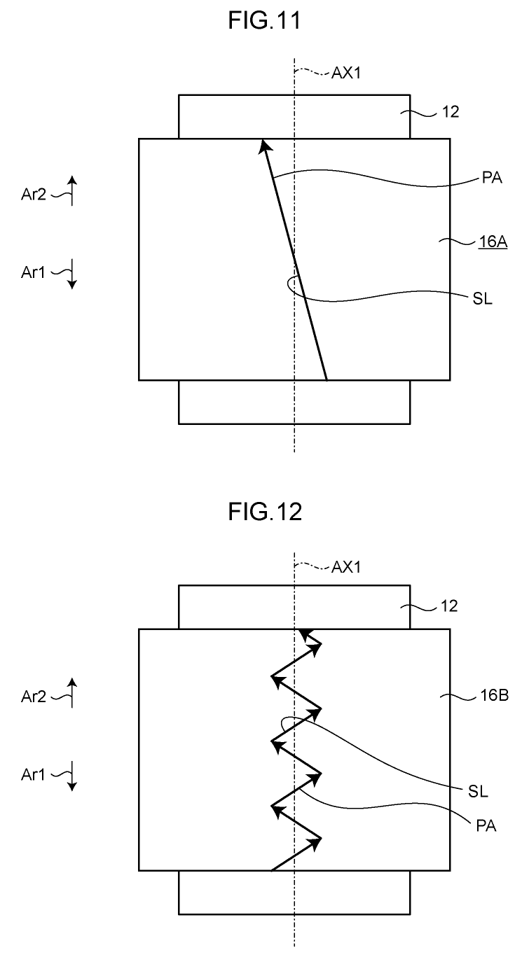

FIGS. 9A, 9B and 9C and FIG. 11 are diagrams each explaining the first modification of the embodiment. Specifically, FIGS. 9A, 9B and 9C are diagrams corresponding to the diagrams illustrated in FIGS. 5A, 5B and 5C, and is a diagram illustrating an assembly order of the first support 16 according to the first modification with respect to the vibration transmitter 12. FIG. 10 is a diagram corresponding to the diagram illustrated in FIG. 6. FIG. 11 is a diagram corresponding to the diagrams illustrated in FIG. 7 and FIG. 8.

In the above described embodiment, it may be possible to adopt a first support 16A according to the first modification illustrated in FIGS. 9A, 9B and 9C to FIG. 11 instead of adopting the first support 16.

The first support 16A is made of the same resin material as that used for the first support 16 that has been explained above in the embodiment. More specifically, the first support 16A has, as indicated by FIG. 9B, substantially the same inner diameter dimension as the outer diameter dimension of the groove 122, and has a ring shape into which the vibration transmitter 12 is inserted. Moreover, it is preferable that the inner diameter dimension of the first support 16A is constituted to have a dimension that is smaller than the outer diameter dimension of the groove 122 in order to constitute the structure in which a gap is not formed between the first support 16A and the vibration transmitter 12. In addition, the slit SL that extends in a direction from one end to the other end along the central axis Ax1 is provided at the first support 16A (FIG. 9B).

Then, the first support 16A is assembled into the groove 122.

Specifically, the worker arranges the vibration transmitter 12 on an inner side of the first support 16A while pushing out both edge portions in the circumferential direction corresponding to a boundary bounded by the slit SL that is provided in the first support 16A in a direction away from each other (FIG. 9C).

As described above, the outer peripheral surface of the first support 16A that is assembled in the vibration transmitter 12 is contact with the inner peripheral surface of the first inner tube TI1, as illustrated in FIG. 10. As a result of this, the fluid that has entered from the distal end of the inner pipe PI into the gap between the first inner tube TI1 and the vibration transmitter 12 is inhibited from flowing toward the proximal end side Ar2 by the first support 16A. Specifically, when the fluid flows by way of the first support 16A, the fluid follows the slit SL (FIG. 11). In other words, the slit SL functions as the path PA for the fluid (FIG. 11). In the present embodiment, the slit SL linearly extends, and also, intersects the direction along the central axis Ax1.

Even in a case where the first support 16A is configured by use of a single member as explained above in the first modification, the same effects similar to those described above in the embodiment is provided.

### Second Modification

FIG. 12 is a diagram explaining the second modification of the embodiment. Specifically, FIG. 12 is a diagram corresponding to the diagram illustrated in FIG. 11.

In the first modification, the slit SL linearly extends from one end toward the other end of the first support 16A in a direction along the central axis Ax1, but the configuration is not limited to this example. As the slit SL, as with a first support 16B according to the second modification illustrated in FIG. 12, it may be possible to adopt a shape that extends in a zig-zag manner from one end toward the other end of the first support 16B in the direction along the central axis Ax1. In other words, the slit SL according to the second modification functions as the path PA for the fluid (FIG. 12), but extends in an intersected state in a direction along the central axis Ax1.

Even in a case where the first support 16B is configured by use of a single member as explained above in the second modification, the same effects similar to those described above in the embodiment is provided.

### Third Modification

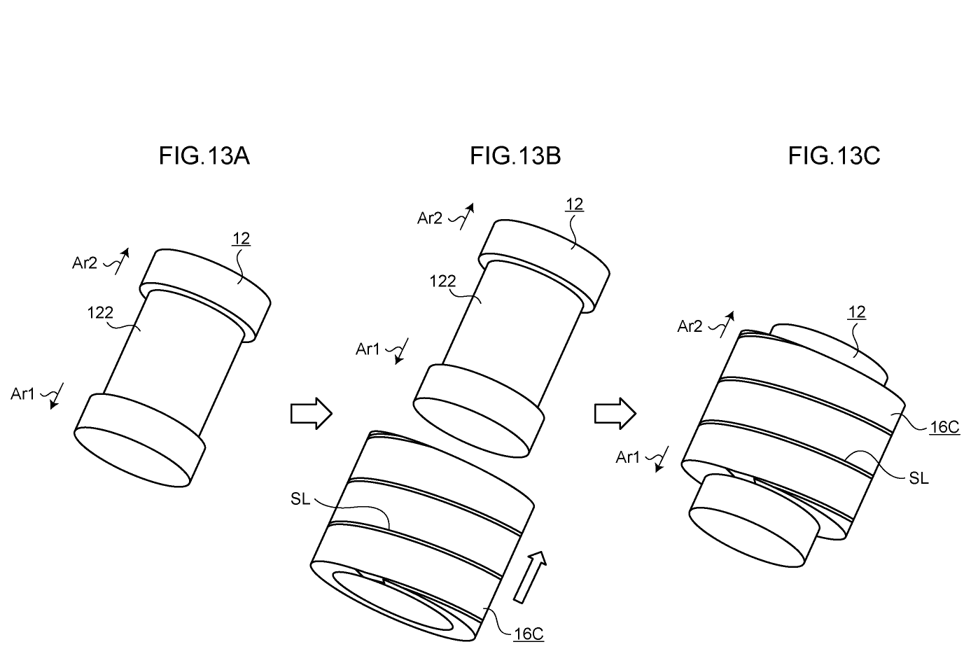

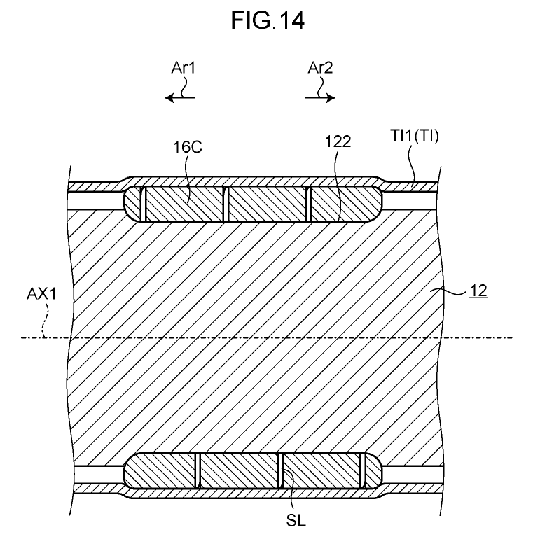

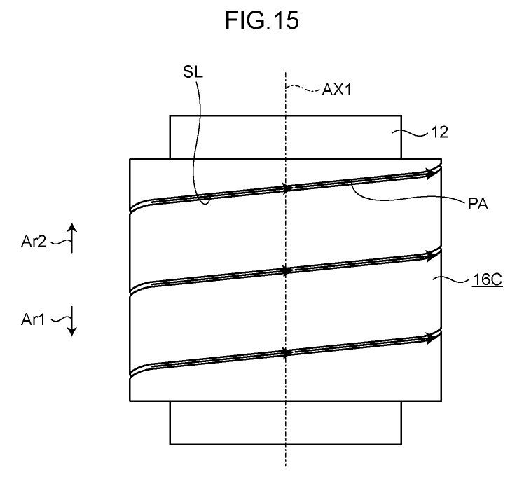

FIGS. 13A, 13B and 13C to FIG. 15 are diagrams each explaining the third modification of the embodiment. Specifically, FIGS. 13A, 13B and 13C are diagrams corresponding to the diagrams illustrated in FIGS. 9A, 9B and 9C, and is a diagram illustrating an assembly order of a first support 16C according to the third modification with respect to the vibration transmitter 12. FIG. 14 is a diagram corresponding to the diagram illustrated in FIG. 10. FIG. 15 is a diagram corresponding to the diagram illustrated in FIG. 11.

In the above described first modification, the slit SL linearly extends from one end to the other end of the first support 16A in the direction along the central axis Ax1, but the configuration is not limited to this example. As the slit SL, as with the first support 16C according to the third modification illustrated in FIGS. 13A, 13B and 13C to FIG. 15, it may be possible to adopt a shape that extends in a spiral manner from one end toward the other end of the first support 16C in the direction along the central axis Ax1. In other words, the slit SL according to the third modification functions as a flow path PA for the fluid (FIG. 15), but extends in an intersected state in a direction along the central axis Ax1.

Even in a case where the first support 16C is configured by use of a single member as explained above in the third modification, the same effects similar to those described above in the embodiment is provided.

### Fourth Modification

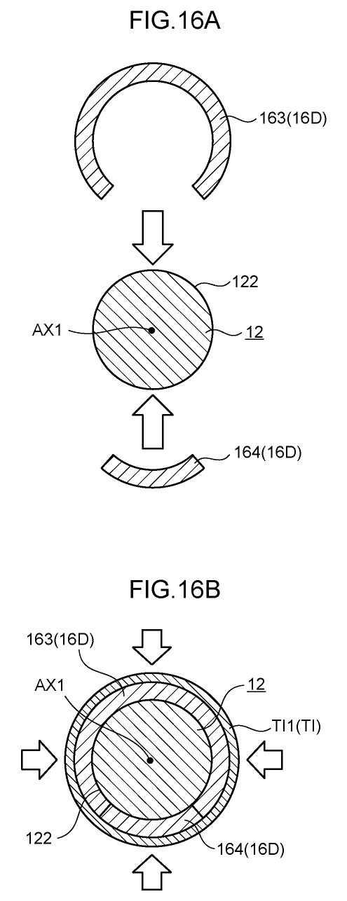

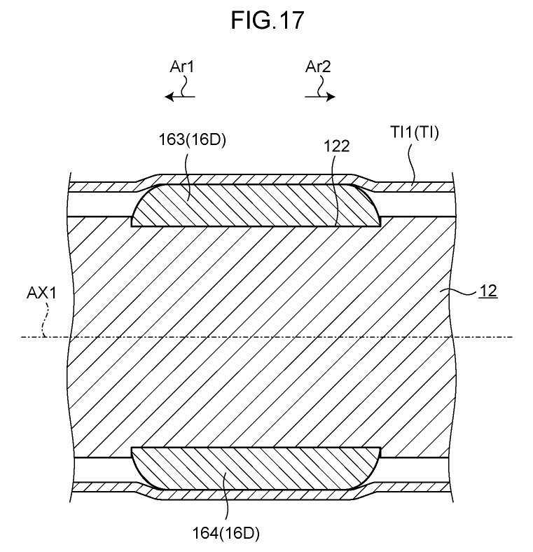

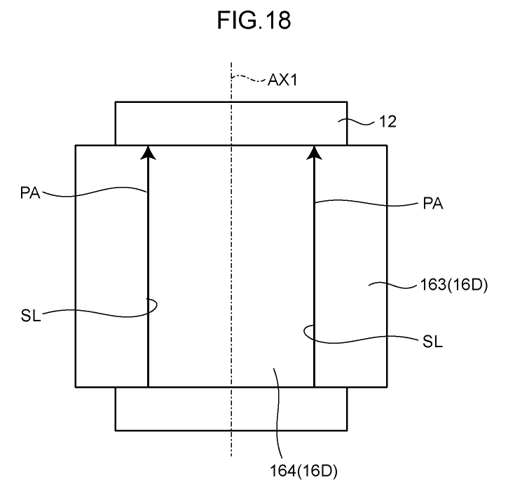

FIGS. 16A and 16B to FIG. 18 are diagrams each explaining the fourth modification of the embodiment. Specifically, FIGS. 16A and 16B are diagrams illustrating an assembly order of a first support 16D according to the fourth modification with respect to the vibration transmitter 12. Moreover, FIGS. 16A and 16B are diagrams viewed along the central axis Ax1. FIG. 17 is a diagram corresponding to the diagram illustrated in FIG. 6. FIG. 18 is a diagram corresponding to each of the diagrams illustrated in FIG. 7 and FIG. 8.

In the above described embodiment, it may be possible to adopt the first support 16D according to the fourth modification illustrated in FIGS. 16A and 16B to FIG. 18 instead of adopting the first support 16.

The first support 16D is made of the same resin material as that used for the first support 16 that has been explained above in the embodiment. More specifically, the first support 16D has, as illustrated in FIGS. 16A and 16B and FIG. 17, substantially the same inner diameter dimension as the outer diameter dimension of the groove 122, and has a ring shape into which the vibration transmitter 12 is inserted. Moreover, it is preferable that the inner diameter dimension of the first support 16D is constituted to have a dimension that is smaller than the outer diameter dimension of the groove 122 in order to constitute the structure in which a gap is not formed between the first support 16D and the vibration transmitter 12. The first support 16D is divided into two portions along the circumferential direction around the central axis of the first support 16D. One of the two portions is a C ring 163 having a C-shape. Further, the other of the two portions is a lid 164 that blocks a notch 1631 formed in the C ring 163.

Then, the first support 16D is assembled, as described below, into the groove 122.

Specifically, first, the worker arranges the vibration transmitter 12 on an inner side of the C-shape formed as the C ring 163 while pushing out both end portions of the C ring 163 in the circumferential direction (both edge portions of the notch 1631). Then, the worker fits the lid 164 into the notch 1631. After that, the C ring 163 and the lid 164 are arranged, as indicated by FIG. 16B, in the vibration transmitter 12 in a close contact state due to thermal contraction of the first inner tube TI1 that is the heat shrink tube.

As explained above, the outer peripheral surface of the first support 16D that has been assembled in the vibration transmitter 12 is contact with the inner peripheral surface of the first inner tube TI1, as indicated by FIG. 16B and as illustrated in FIG. 17. As a result of this, the fluid that has entered from the distal end of the inner pipe PI into the gap between the first inner tube TI1 and vibration transmitter 12 is inhibited from flowing toward the proximal end side Ar2 by the first support 16D. Specifically, when the fluid flows by way of the first support 16D, the fluid follows the boundary portions SL between the C ring 163 and the lid 164 (FIG. 18). Each of the boundary portions SL extends from one end toward the other end of the first support 16D in the direction along the central axis Ax1, and corresponds to the slit. In other words, each of the boundary portions SL functions as the path PA for the fluid (FIG. 18). In the present embodiment, the boundary portions SL do not intersect the direction along the central axis Ax1, but, as described above, in a state in which the gap therebetween is narrowed by the first inner tube TI1.

Even in a case where the configuration of the first support 16D according to the above explained fourth modification is adopted, the same effects similar to those described above in the embodiment and the eighth modification are provided.

With the ultrasound treatment tool according to the disclosure, it is possible to ensure watertightness while suppressing a deflection of a vibration transmitter.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An ultrasound treatment tool comprising:
a vibration transmitter configured to transmit ultrasound vibration from a proximal end toward a distal end;
a tube into which the vibration transmitter is inserted; and
a rigid part that is provided on an outer peripheral surface of the vibration transmitter at a node position of the ultrasound vibration, the rigid part being in contact with an inner peripheral surface of the tube, the rigid part being made of a resin material that is harder than the tube, the rigid part being a pair of C rings each having a C-shape into which the vibration transmitter is inserted, each C ring including a C ring main body having a C-shape in which a notch is formed, and a protrusion protruding from the C ring main body along a longitudinal direction of the vibration transmitter, the pair of C rings being attached to the vibration transmitter in a state in which the protrusion of one of the pair of C rings is arranged at the notch of another one of the pair of C rings.

2. The ultrasound treatment tool according to claim 1, wherein the pair of C rings have a same shape.

3. The ultrasound treatment tool according to claim 1, wherein the rigid part is provided at least one of the node position that is located closest to the distal end or the node position that is located second closest to the distal end from among node positions of the ultrasound vibration on the outer peripheral surface of the vibration transmitter.

4. The ultrasound treatment tool according to claim 1, wherein the rigid part is made of a resin material that is one of polytetrafluoroethylene, polyether ether ketone, polyphenylsulfone, and polyoxymethylene.

5. The ultrasound treatment tool according to claim 1, wherein the rigid part has a circular ring shape, and includes a slit that extends from one end toward another end of the rigid part in a longitudinal direction of the vibration transmitter.

6. The ultrasound treatment tool according to claim 5, wherein at least a part of the slit intersects the longitudinal direction of the vibration transmitter.

7. The ultrasound treatment tool according to claim 5, wherein the slit extends in a zig-zag manner from the one end toward the other end of the rigid part.

8. The ultrasound treatment tool according to claim 5, wherein the slit extends in a spiral manner from the one end toward the other end of the rigid part.

9. The ultrasound treatment tool according to claim 1, wherein a groove in which the rigid part is attached is provided at the node position that is located closest to the distal end from among node positions of the ultrasound vibration on the outer peripheral surface of the vibration transmitter.

10. The ultrasound treatment tool according to claim 1, further comprising:
a jaw configured to be opened and closed with respect to the vibration transmitter; and
a pipe configured to cover an outer peripheral surface of the tube,
wherein the jaw is configured to be opened and closed with respect to the vibration transmitter depending on a forward and backward movement of the pipe along a longitudinal direction of the vibration transmitter.

11. The ultrasound treatment tool according to claim 10, further comprising an outer pipe configured to cover an outer peripheral surface of the pipe.

12. The ultrasound treatment tool according to claim 10, wherein an electrode for applying high frequency current between the vibration transmitter and the jaw is provided in each of the vibration transmitter and the jaw.

13. The ultrasound treatment tool according to claim 10, wherein an operation handle for opening and closing the jaw with respect to the vibration transmitter in accordance with an operation of a user is provided on a proximal end side of the pipe.
